(19) United States
(12) Patent Application Publication
(10) Pub. No.: US 2010/0152299 A1
(43) Pub. Date: Jun. 17, 2010

(54) Process for making compositions with poorly compressible therapeutic compounds

(76) Inventors: Madhav Vasanthavada, Basking Ridge, NJ (US); Jay Lakshman, Cedar Knolls, NJ (US); Wein-Qin Tong, Basking Ridge, NJ (US); Abu T. Serajuddin, Flushing, NY (US); Yatindra Joshi, Princeton, NJ (US); James Kowalsi, Belle Mead, NJ (US)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 11/909,509

(22) PCT Filed: May 8, 2006
(86) PCT No.: PCT/US06/17708

§ 371 Date: Sep. 24, 2007

Related U.S. Application Data
(60) Provisional application No. 60/679,587, filed on May 10, 2005.
(60) Provisional application No. 60/693,155, filed on Jun. 23, 2005.

(51) Int. Cl. A61K 31/155; B29B 9/10

(52) U.S. Cl. 514/632; 264/13

(57) ABSTRACT

A process for preparing solid dosage forms that contain poorly compressible therapeutic compound. The process, for example, provides for the inventive use of an extruder, especially a twin screw extruder, to melt granulate a therapeutic compound(s) with a granulation excipient.

### FIELD OF THE INVENTION

The present invention relates to a process for making solid oral dosage forms of a poorly compressible and/or a moisture sensitive therapeutic compound. The process features the use of melt granulation with an extruder.

### BACKGROUND OF THE INVENTION

Poor compressibility can impact the ability of formulating a therapeutic compound into a solid oral dosage form, e.g., a tablet. Conventional tablet formulations containing poorly compressible therapeutic compounds often lack adequate hardness and are friable. Thus, special formulation techniques are used to formulate poorly compressible therapeutic compounds into commercially viable solid oral dosage forms, especially tablets.

One way to overcome the poor compressibility of therapeutic compounds is to utilize wet granulation techniques to prepare the tablet formulation. This involves additional unit operations of wet milling, drying and milling of dried granulation. However, tablets prepared by wet methods can show incremental hardness as a function of time and storage temperature. Therefore, tablets prepared by wet methods can show variable product performance. Additionally, certain therapeutic compounds are susceptible to degradation when in contact with water; thus, wet granulation with water may not be ideal.

Thus, there is a need for a method of preparing pharmaceutical compositions of poorly compressible therapeutic compounds that have adequate hardness with good reproducibility. This invention addresses that need by utilizing melt granulation techniques. A particularly inventive aspect of the present invention is the use of an extruder to provide for melt granulation compounding.

Traditionally, extruders at elevated temperatures in a pharmaceutical context have been used for the manufacture of solid dispersion and/or solid solutions that have required at least a partial melting of the therapeutic compound. Surprisingly, it has been found that the use of extruders can be useful in the preparation of melt granulated solid dosage forms without the need for the melting of the therapeutic compound.

### SUMMARY OF THE INVENTION

The present invention features a process for making a pharmaceutical composition that includes the steps of combining a poorly compressible and/or moisture sensitive therapeutic compound with at least one granulation excipient to form a mixture; blending or kneading the mixture in an extruder, e.g., a twin screw extruder, while heating the mixture to a temperature less than the melting point or melting range of the therapeutic compound; and extruding the mixture through an optional die to form an extrudate.

In a particular aspect, the extrudate can be optionally milled into granules and subsequently compressed using conventional means into a solid oral dosage form. In another aspect of the present invention, the granulation excipient is a polymer having a glass transition temperature that is less than the melting point of the therapeutic compound. Particularly useful polymers include water-soluble, water-swellable and water insoluble polymers.

The inventive process of the present invention can be used to make both immediate release and sustained release pharmaceutical compositions.

### DETAILED DESCRIPTION OF THE INVENTION

The present invention relates to a process for preparing pharmaceutical compositions of poorly compressible and/or moisture sensitive therapeutic compounds. The inventive process features melt granulation, using an extruder, of a poorly compressible therapeutic compound with a granulation excipient. The melt granulation of the poorly compressible therapeutic compound is accomplished without the need for any melting of the therapeutic compound.

As used herein the term “pharmaceutical composition” means a mixture containing a therapeutic compound to be administered to a mammal, e.g., a human in order to prevent, treat or control a particular disease or condition affecting the mammal.

As used herein the term “pharmaceutically acceptable” refers to those compounds, materials, compositions and/or dosage forms, which are, within the scope of sound medical judgment, suitable for contact with the tissues of mammals, especially humans, without excessive toxicity, irritation, allergic response and other problem complications commensurate with a reasonable benefit/risk ratio.

As used herein the term “therapeutic compound” means any compound, substance, drug, medicament, or active ingredient having a therapeutic or pharmacological effect, and which is suitable for administration to a mammal, e.g., a human, in a composition that is particularly suitable for oral administration.

As used herein the term “poorly compressible” therapeutic compound refers to a compound that does not easily bond to form a tablet upon the application of a force. A tablet produced solely of the therapeutic compound weighing one gram and compressed under a force ranging from 5 kN to 25 kN with a dwell time under thirty seconds, would provide friability at or above an acceptable limit of 1.0% (w/w) when tablets weighing approximately ten grams (or at least ten units) are tested after five hundred drops immediately after compression. Such compounds may require additional processing and special formulating, for example wet granulating or roller compacting, prior to compression. High dosages of a therapeutic compound may also render a therapeutic compound not appropriate for direct compression because of poor flowability and poor compressibility.

As used herein, the term “moisture-sensitive” therapeutic compound refers to a therapeutic compound which undergoes spontaneous degradation, e.g., by hydrolysis of at least 1% by weight of the therapeutic compound when the therapeutic compound contacts water.

Examples of therapeutic classes of therapeutic compounds include, but are not limited to, antacids, anti-inflammatory substances, coronary dilators, cerebral dilators, peripheral vasodilators, anti-infectives, psychotropics, antimanics, stimulants, antihistamines, anti-cancer therapeutic compounds, laxatives, decongestants, vitamins, gastrointestinal sedatives, antidiarrheal preparations, anti-anginal therapeutic compounds, vasodilators, antiarrythmics, anti-hypertensive therapeutic compounds, vasoconstrictors and migraine treatments, anticoagulants and antithrombotic therapeutic compounds, analgesics, anti-pyretics, hypnotics, sedatives, anti-emetics, anti-nauseants, anti-convulsants, neuromuscular therapeutic compounds, hyper- and hypoglycemic agents, thyroid and anti-thyroid preparations, diuretics, anti-spasmodics, uterine relaxants, mineral and nutritional additives, anti-obesity therapeutic compounds, anabolic therapeutic compounds, erythropoietic therapeutic compounds, anti-asthmatics, expectorants, cough suppressants, mucolytics, anti-uricemic therapeutic compounds, and therapeutic compounds or substances acting locally in the mouth.

Exemplary therapeutic compounds include, but are not limited to, gastrointestinal sedatives, such as metoclopramide and propantheline bromide; antacids, such as aluminum trisilicate, aluminum hydroxide and cimetidine; anti-inflammatory therapeutic compounds, such as phenylbutazone, indomethacin, naproxen, ibuprofen, flurbiprofen, diclofenac, dexamethasone, prednisone and prednisolone; coronary vasodilator therapeutic compounds, such as glyceryl trinitrate, isosorbide dinitrate and pentaerythritol tetranitrate; peripheral and cerebral vasodilators, such as soloctidilum, vincamine, naftidrofuryl oxalate, co-dergocrine mesylate, cyclandelate, papaverine and nicotinic acid; anti-infective therapeutic compounds, such as erythromycin stearate, cephalexin, nalidixic acid, tetracycline hydrochloride, ampicillin, flucolaxacillin sodium, hexamine mandelate and hexamine hippurate; neuroleptic therapeutic compounds, such as fluazepam, diazepam, temazepam, amitryptyline, doxepin, lithium carbonate, lithium sulfate, chlorpromazine, thioridazine, trifluperazine, fluphenazine, piperothiazine, haloperidol, maprotiline hydrochloride, imipramine and desmethylimipramine; central nervous stimulants, such as methylphenidate, ephedrine, epinephrine, isoproterenol, amphetamine sulfate and amphetamine hydrochloride; anti-histamic therapeutic compounds such as diphenhydramine, diphenylpyraline, chlorpheniramine and brompheniramine; anti-diarrheal therapeutic compounds, such as bisacodyl and magnesium hydroxide; laxative therapeutic compounds, such as dioctyl sodium sulfosuccinate; nutritional supplements, such as ascorbic acid, alpha tocopherol, thiamine and pyridoxine; anti-spasmotic therapeutic compounds, such as dicyclomine and diphenoxylate; therapeutic compounds effecting the rhythm of the heart, such as verapamil, nifedepine, diltiazem, procainamide, disopyramide, bretylium tosylate, quinidine sulfate and quinidine gluconate; therapeutic compounds used in the treatment of hypertension, such as propranolol hydrochloride, guanethidine monosulphate, methyldopa, oxprenolol hydrochloride, captopril and hydralazine; therapeutic compounds used in the treatment of migraine, such as ergotamine; therapeutic compounds effecting coagulation of blood, such as epsilon aminocaproic acid and protamine sulfate; analgesic therapeutic compounds, such as acetylsalicylic acid, acetaminophen, codeine phosphate, codeine sulfate, oxycodone, dihydrocodeine tartrate, oxycodeinone, morphine, heroin, nalbuphine, butorphanol tartrate, pentazocine hydrochloride, cyclazacine, pethidine, buprenorphine, scopolamine and mefenamic acid; anti-epileptic therapeutic compounds, such as phenyloin sodium and sodium valproate; neuromuscular therapeutic compounds, such as dantrolene sodium; therapeutic compounds used in the treatment of diabetes, such as metformin, tolbutamide, diabenase glucagon and insulin; therapeutic compounds used in the treatment of thyroid gland dysfunction, such as triiodothyronine, thyroxine and propylthiouracil; diuretic therapeutic compounds, such as furosemide, chlorthalidone, hydrochlorthiazide, spironolactone and triampterene; uterine relaxant therapeutic compounds, such as ritodrine; appetite suppressants, such as fenfluramine hydrochloride, phentermine and diethylproprion hydrochloride; anti-asthmatic therapeutic compounds, such as aminophylline, theophylline, salbutamol, orciprenaline sulphate and terbutaline sulphate, expectorant therapeutic compounds, such as guaiphenesin; cough suppressants, such as dextromethorphan and noscapine; mucolytic therapeutic compounds, such as carbocisteine; anti-septics, such as cetylpyridinium chloride, tyrothricin and chlorhexidine; decongestant therapeutic compounds, such as phenylpropanolamine and pseudoephedrine; hypnotic therapeutic compounds, such as dichloralphenazone and nitrazepam; anti-nauseant therapeutic compounds, such as promethazine theoclate; haemopoetic therapeutic compounds, such as ferrous sulphate, folic acid and calcium gluconate, uricosuric therapeutic compounds, such as sulphinpyrazone, allopurinol and probenecid and the like.

The poorly compressible therapeutic compound(s) is present in the pharmaceutical compositions of the present invention in a therapeutically effective amount or concentration. Such a therapeutically effective amount or concentration is known to one of ordinary skill in the art as the amount or concentration varies with the therapeutic compound being used and the indication which is being addressed. For example, in accordance with the present invention, the therapeutic compound may be present in an amount by weight of about 0.05% to about 99% weight of pharmaceutical composition. In one embodiment, the therapeutic compound may be present in an amount by weight of about 10% to about 95% by weight of the pharmaceutical composition.

As used herein, the term “immediate release” refers to the rapid release of the majority of the therapeutic compound, e.g., greater than about 50%, about 60%, about 70%, about 80%, or about 90% within a relatively short time, e.g., within 1 hour, 40 minutes, 30 minutes or 20 minutes after oral ingestion. Particularly useful conditions for immediate-release are release of at least or equal to about 80% of the therapeutic compound within thirty minutes after oral ingestion. The particular immediate release conditions for a specific therapeutic compound will be recognized or known by one of ordinary skill in the art.

As used herein, the term “sustained release”, or “modified release”, refers to the gradual but continuous or sustained release over a relatively extended period of the therapeutic compound content after oral ingestion. The release will continue over a period of time and may continue through until and after the pharmaceutical composition reaches the intestine. Sustained release may also refer to delayed release in which release of the therapeutic compound does not start immediately when the pharmaceutical composition reaches the stomach but is delayed for a period of time, for instance, until when the pharmaceutical composition reaches the intestine when the increasing pH is used to trigger release of the therapeutic compound from the pharmaceutical composition.

As used herein the term “granulation excipient” refers to any pharmaceutically acceptable material or substance that can be melt granulated with the poorly compressible therapeutic compound as further described below. The granulation excipient, for example, can be a polymer or a non-polymeric material.

As used herein the term “polymer” refers to a polymer or mixture of polymers that have a glass transition temperature, softening temperature or melting temperature by itself or in combination not exceeding the melting point (or melting range) of the poorly compressible therapeutic compound. The glass transition temperature (“Tg”) is the temperature at which such polymer's characteristics change from that of highly viscous to that of relatively less viscous mass. Types of polymers include, but are not limited to, water-soluble, water-swellable, water insoluble polymers and combinations of the foregoing.

Examples of polymers include, but are not limited to:

homopolymers and copolymers of N-vinyl lactams, e.g., homopolymers and copolymers of N-vinyl pyrrolidone (e.g., polyvinylpyrrolidone), copolymers of N-vinyl pyrrolidone and vinyl acetate or vinyl propionate;

cellulose esters and cellulose ethers (e.g., methylcellulose and ethylcellulose) hydroxyalkylcelluloses (e.g., hydroxypropylcellulose), hydroxyalkylalkylcelluloses (e.g., hydroxypropylmethylcellulose), cellulose phthalates (e.g., cellulose acetate phthalate and hydroxylpropylmethylcellulose phthalate) and cellulose succinates (e.g., hydroxypropylmethylcellulose succinate or hydroxypropylmethylcellulose acetate succinate);

high molecular polyalkylene oxides such as polyethylene oxide and polypropylene oxide and copolymers of ethylene oxide and propylene oxide;

polyacrylates and polymethacrylates (e.g., methacrylic acid/ethyl acrylate copolymers, methacrylic acid/methyl methacrylate copolymers, butyl methacrylate/2-dimethylaminoethyl methacrylate copolymers, poly(hydroxyalkyl acrylates), poly(hydroxyalkyl methacrylates));

polyacrylamides;

vinyl acetate polymers such as copolymers of vinyl acetate and crotonic acid, partially hydrolyzed polyvinyl acetate;

polyvinyl alcohol; and

oligo- and polysaccharides such as carrageenans, galactomannans and xanthan gum, or mixtures of one or more thereof.

As used herein, the term “plasticizer” refers to a material that may be incorporated into the pharmaceutical composition in order to decrease the glass transition temperature and the melt viscosity of a polymer by increasing the free volume between polymer chains. Plasticizers, for example, include, but are not limited to, water; citrate esters (e.g., triethylcitrate, triacetin); low molecular weight poly(alkylene oxides) (e.g., poly(ethylene glycols), poly(propylene glycols), poly(ethylene/propylene glycols)); glycerol, pentaerythritol, glycerol monoacetate, diacetate or triacetate; propylene glycol; sodium diethyl sulfosuccinate; and the therapeutic compound itself. The plasticizer can be present in concentration from about 0% to 15%, e.g., 0.5% to 5% by weight of the pharmaceutical composition. Examples of plasticizers can also be found in The Handbook of Pharmaceutical Additives, Ash et al., Gower Publishing (2000).

Non-polymeric granulation excipients include, but are not limited to, esters, hydrogenated oils, oils, natural waxes, synthetic waxes, hydrocarbons, fatty alcohols, fatty acids, monoglycerides, diglycerides, triglycerides and mixtures thereof.

Examples of esters, such as glyceryl esters include, but are not limited to, glyceryl monostearate, e.g., CAPMUL GMS from Abitec Corp. (Columbus, Ohio); glyceryl palmitostearate; acetylated glycerol monostearate; sorbitan monostearate, e.g., ARLACEL 60 from Uniqema (New Castle, Del.); and cetyl palmitate, e.g., CUTINA CP from Cognis Corp. (Düsseldorf, Germany), magnesium stearate and calcium stearate.

Examples of hydrogenated oils include, but are not limited to, hydrogenated castor oil; hydrogenated cottonseed oil; hydrogenated soybean oil; and hydrogenated palm oil. An example of oil include sesame oil.

Examples of waxes include, but are not limited to, carnauba wax, beeswax and spermaceti wax. Examples of hydrocarbons include, but are not limited to, microcrystalline wax and paraffin. Examples of fatty alcohols, i.e., higher molecular weight nonvolatile alcohols that have from about 14 to about 31 carbon atoms include, but are not limited to, cetyl alcohol, e.g., CRODACOL C-70 from Croda Corp. (Edison, N.J.); stearyl alcohol, e.g., CRODACOL S-95 from Croda Corp; lauryl alcohol; and myristyl alcohol. Examples of fatty acids which may have from about 10 to about 22 carbon atoms include, but are not limited to, stearic acid, e.g., HYSTRENE 5016 from Crompton Corp. (Middlebury, Conn.); decanoic acid; palmitic acid; lauric acid; and myristic acid.

As used herein, the term “melt granulation” refers to the following compounding process that comprises the steps of:
(a) forming a mixture of a poorly compressible therapeutic compound with at least one granulation excipient;
(b) granulating the mixture using an extruder while heating the mixture to a temperature that is less than or about at the melting point (or melting range) of the poorly compressible therapeutic compound; and
(c) cooling the extrudate to room temperature, for example, at a controlled rate.

The heating and mixing of the therapeutic compound and the granulation excipient to form an internal phase of granules (i.e., from the extrudate) is accomplished by the use of an extruder. The granulation excipient, e.g., can be present in an amount from about 1% to about 50% by weight of the composition. In one embodiment, the granulation excipient may be present in an amount from about 3 to about 25% by weight of the composition. The therapeutic compound may be present in an amount from about 50% to about 99% by weight of the composition. In one embodiment, the therapeutic compound may be present in an amount of about 60% to about 97%. Unlike granules made during a wet granulation process, the melt granulation process of the present invention does not necessarily require a granulation fluid, for example, water, methanol, ethanol, isopropanol or acetone during the granulation process.

The resulting granules are, for example, particles of the therapeutic compound coated or substantially coated by the granulation excipient, or alternatively, particles of the therapeutic compound embedded or substantially embedded with or within the granulation excipient.

In general, an extruder includes a rotating screw(s) within a stationary barrel with an optional die located at one end of the barrel. Along the entire length of the screw, distributive kneading of the materials (e.g., the therapeutic compound, release retarding material, and any other needed excipients) is provided by the rotation of the screw(s) within the barrel. Conceptually, the extruder can be divided into at least three sections: a feeding section; a heating section and a metering section. In the feeding section, the raw materials are fed into the extruder, e.g. from a hopper. In the heating section, the raw materials are heated to a temperature less than the melting temperature of the poorly compressible therapeutic compound. After the heating section is a metering section in which the mixed materials are extruded through an optional die into a particular shape, e.g., granules or noodles. Types of extruders particularly useful in the present invention are single-, twin- and multi-screw extruders, optionally configured with kneading paddles.

Once the granules are obtained, the granules may be formulated into oral forms, e.g., solid oral dosage forms, such as tablets, pills, lozenges, caplets, capsules or sachets, by adding additional conventional excipients which comprise an external phase of the pharmaceutical composition. The external phase of the pharmaceutical composition can also comprise an additional therapeutic compound. Such solid oral dosage forms, e.g., are unit oral dosage forms. Examples of such excipients include, but are not limited to, release retardants, plasticizers, disintegrants, binders, lubricants, glidants, stabilizers, fillers and diluents. One of ordinary skill in the art may select one or more of the aforementioned excipients with respect to the particular desired properties of the solid oral dosage form by routine experimentation and without any undue burden. The amount of each excipient used may vary within ranges conventional in the art. The following references which are all hereby incorporated by reference discloses techniques and excipients used to formulate oral dosage forms. See The Handbook of Pharmaceutical Excipients, 4^{th} edition, Rowe et al., Eds., American Pharmaceuticals Association (2003); and Remington: the Science and Practice of Pharmacy, 20^{th} edition, Gennaro, Ed., Lippincott Williams & Wilkins (2003).

As used herein the term “release retardant” refers to any material or substance that slows the release of a therapeutic compound from a pharmaceutical composition when orally ingested. Various sustained release systems, as known in the art, can be accomplished by the use of a release retarding component, e.g., a diffusion system, a dissolution system and/or an osmotic system. A release retardant can be polymeric or non-polymeric in nature. The pharmaceutical compositions of the present invention can include, for example, at least five percent of a release retardant by weight of the composition if a sustained release composition is desired.

Examples of pharmaceutically acceptable disintegrants include, but are not limited to, starches; clays; celluloses; alginates; gums; cross-linked polymers, e.g., cross-linked polyvinyl pyrrolidone or crospovidone, e.g., POLYPLASDONE XL from International Specialty Products (Wayne, N.J.); cross-linked sodium carboxymethylcellulose or croscarmellose sodium, e.g., AC-DI-SOL from FMC; and cross-linked calcium carboxymethylcellulose; soy polysaccharides; and guar gum. The disintegrant may be present in an amount from about 0% to about 10% by weight of the composition. In one embodiment, the disintegrant is present in an amount from about 0.1% to about 1.5% by weight of composition.

Examples of pharmaceutically acceptable binders include, but are not limited to, starches; celluloses and derivatives thereof, for example, microcrystalline cellulose, e.g., AVICEL PH from FMC (Philadelphia, Pa.), hydroxypropyl cellulose hydroxylethyl cellulose and hydroxylpropylmethyl cellulose METHOCEL from Dow Chemical Corp. (Midland, Mich.); sucrose; dextrose; corn syrup; polysaccharides; and gelatin. The binder may be present in an amount from about 0% to about 50%, e.g., 10-40% by weight of the composition.

Examples of pharmaceutically acceptable lubricants and pharmaceutically acceptable glidants include, but are not limited to, colloidal silica, magnesium trisilicate, starches, talc, tribasic calcium phosphate, magnesium stearate, aluminum stearate, calcium stearate, magnesium carbonate, magnesium oxide, polyethylene glycol, powdered cellulose and microcrystalline cellulose. The lubricant may be present in an amount from about 0% to about 10% by weight of the composition. In one embodiment, the lubricant may be present in an amount from about 0.1% to about 1.5% by weight of composition. The glidant may be present in an amount from about 0.1% to about 10% by weight.

Examples of pharmaceutically acceptable fillers and pharmaceutically acceptable diluents include, but are not limited to, confectioner's sugar, compressible sugar, dextrates, dextrin, dextrose, lactose, mannitol, microcrystalline cellulose, powdered cellulose, sorbitol, sucrose and talc. The filler and/or diluent, e.g., may be present in an amount from about 15% to about 40% by weight of the composition.

To make pharmaceutical compositions of the present invention, a therapeutic compound and a granulation excipient are blended in a ratio in a range of 99:1 to 1:1 (on a dry weight basis) prior to, or upon addition into the hopper of an extruder. In one exemplary embodiment, this ratio between the therapeutic compound and granulation excipient can be in a range of 97:3 to 60:40 (on a dry weight basis). Yet in another alternative embodiment, the ratio can be in a range of 97:3 to 75:25 (on a dry weight basis). Optionally, a plasticizer can be added to the internal phase.

The mixture is heated to a temperature(s) less than the melting temperature of the therapeutic compound. As the mixture is being heated, it is also being kneaded by the screw(s) of the extruder. The mixture is maintained at the elevated temperature and blended for a time sufficient to form a granulated product. After the mixture is conveyed down the entire length of the barrel, a granulated product (being the extrudate) is obtained, and the granulated mixture is cooled.

After cooling, the extrudate can be milled and subsequently screened through a sieve. The granules (which constitute the internal phase of the pharmaceutical composition) are then combined with solid oral dosage form excipients (the external phase of the pharmaceutical composition), i.e., fillers, binders, disintegrants, lubricants and etc. The combined mixture may be further blended, e.g., through a V-blender, and subsequently compressed or molded into a tablet, for example a monolithic tablet, or encapsulated by a capsule.

Once the tablets are obtained, they can be optionally coated with a functional or non-functional coating as known in the art. Examples of coating techniques include, but are not limited to, sugar coating, film coating, microencapsulation and compression coating. Types of coatings include, but are not limited to, enteric coatings, sustained release coatings, controlled-release coatings.

The utility of all the pharmaceutical compositions of the present invention may be observed in standard clinical tests in, for example, known indications of drug dosages giving therapeutically effective blood levels of the therapeutic compound; for example using dosages in the range of 2.5-1000 mg of therapeutic compound per day for a 75 kg mammal, e.g., adult and in standard animal models.

The present invention provides a method of treatment of a subject suffering from a disease, condition or disorder treatable with a therapeutic compound comprising administering a therapeutically effective amount of a pharmaceutical composition of the present invention to a subject in need of such treatment.

The following examples are illustrative, but do not serve to limit the scope of the invention described herein. The examples are meant only to suggest a method of practicing the present invention.

An example of a therapeutic compound appropriate for the present invention is metformin hydrochloride. A unit dosage form, e.g., a single tablet or capsule, of metformin hydrochloride, can comprise between 250 mg to 2000 mg of metformin hydrochloride, e.g., 250 mg, 500 mg, 750 mg, 850 mg or 1000 mg of metformin. In the present invention, the metformin hydrocholoride can be present in the internal phase of the final solid oral dosage form.

### Example

| | | |
|---|---|---|
| | Percentage | Amount per |
| Ingredient | (w/w) | tablet (mg) |

Internal phase

| | | |
|---|---|---|
| metformin HCl | 91% | 1000 |
| hydroxypropyl cellulose | 9% | 99 |

External phase

| | | |
|---|---|---|
| magnesium stearate | 1% | 11 |
| Total | | 1110 |

The internal phase ingredients i.e. metformin hydrochloride, and hydroxypropyl cellulose available as KLUCEL EXF from Hercules Chemical Co. (Wilmington, Del.) are combined and blended in a bin blender for about two hundred rotations. The blend is introduced into the feed section, or hopper, of a twin screw extruder. A suitable twin screw extruder is the PRISM 16 mm pharmaceutical twin screw extruder available from Thermo Electron Corp. (Waltham, Mass.).

Located at the end of the twin screw extruder is a die with a bore of approximately three mm. The twin screw extruder is configured with five individual barrel zones, or sections, that can independently adjusted to different parameters. Starting from the hopper to the die, the zones are respectively heated to the following temperatures: 40° C., 110° C., 130° C., 170° C. and 185° C. The temperatures of the heating zones do not exceed the melting temperature of metformin hydrochloride which is approximately 232° C. The screw speed is set to 150 rpm, but can be as high as 400 rpm, and the volumetric feed rate is adjusted to deliver between about 30 to 45 grams of material per minute. The throughput rate can be adjusted from 4 g/min to 80 g/min.

The extrudate, or granules, from the extruder are then cooled to room temperature by allowing them to stand from approximately fifteen to twenty minutes. The cooled granules, are subsequently sieved through an 18 mesh screen (i.e., a one mm screen).

For the external phase, the magnesium stearate is first passed through an 18 mesh. The magnesium stearate is then blended with the obtained granules using a suitable bin blender for approximately sixty rotations. The resulting final blend is compressed into tablets using a conventional rotary tablet press (Manesty Beta Press) using a compression force ranging between 6 kN and 25 kN. The resulting tablets are monolithic and having a hardness ranging from 5 kP to 35 kP. Tablets having hardness ranging from 15 kP to 35 kP resulted in acceptable friability of less than 1.0% w/w after five hundred drops. Moreover, these tablets have a disintegration time of less than equal to twenty minutes with discs at 37° C. in 0.1 N HCl.

In contrast, when the formulation of Example 1 is made into tablets by wet granulation or direct compression, the resulting tablets have a hardness ranging from 3 kP to 7 kP when compressed between 6 kN and 26 kN. Moreover, these tablets resulted in a friability greater than 1% (w/w) after five hundred drops. Thus, the results show that the melt granulation process enhances the compressibility of poorly compressible therapeutic compounds.

It is understood that while the present invention has been described in conjunction with the detailed description thereof that the foregoing description is intended to illustrate and not limit the scope of the invention, which is defined by the scope of the following claims. Other aspects, advantages and modifications are within the scope of the claims.

1. A process for making a pharmaceutical composition comprising the steps of:
combining a poorly compressible therapeutic compound with at least one granulating component to form a mixture;
kneading said mixture in an extruder while heating said mixture to a heating temperature less than a melting point of said therapeutic compound; and
extruding said mixture to form granules.

2. The process of claim 1, further comprising the step of compressing said granules to form a solid oral dosage form.

3. The process of claim 1, wherein said granulating component is a polymer having a Tg less than said melting point.

4. The process of claim 3, wherein said polymer is selected from the group consisting of water-soluble polymers, water-swellable polymers and water-insoluble polymers.

5. The process of claim 4, wherein said heating temperature is greater than or equal to said Tg.

6. The process of claim 3, wherein said mixture further comprises a plasticizer.

7. The process of claim 1, wherein said pharmaceutical composition is an immediate-release composition.

8. The process of claim 1, wherein said pharmaceutical composition is a sustained-release composition.

9. The process of claim 8, wherein said pharmaceutical composition comprises a release retardant.

10. The process of claim 1, wherein said extruding is through a die.

11. The process of claim 1, wherein said extruder is a twin-screw extruder.

12. A process for making a pharmaceutical composition comprising the steps of:
combining a moisture sensitive therapeutic compound with at least one granulating component to form a mixture;
kneading said mixture in an extruder while heating said mixture to a heating temperature less than a melting point of said therapeutic compound; and
extruding said mixture to form granules.

13. A process for improving the friability of a tablet containing a poorly compressible therapeutic compound comprising the steps of:
combining a poorly compressible therapeutic compound with at least one granulating component to form a mixture;
kneading said mixture in an extruder while heating said mixture to a heating temperature less than a melting point of said therapeutic compound
extruding said mixture to form granules; and
compressing said granules into a tablet.

14. Pharmaceutical granules prepared by the process of claim 1.

15. The pharmaceutical granules of claim 14, wherein said therapeutic compound is metformin hydrochloride.

16. A pharmaceutical composition comprising pharmaceutical granules of claim 13 and an additional therapeutic compound.

17. The pharmaceutical granules of claim 15, wherein said metformin hydrochloride is present between 250 mg to 2000 mg.

18. A pharmaceutical composition comprising granules made by the process of claim 1, wherein said pharmaceutical composition has a hardness between 15 kP to 35 kP.

19-21. (canceled)
